(19) United States
(12) Patent Application Publication
(10) Pub. No.: US 2026/0263345 A1
(43) Pub. Date: Sep. 10, 2026

(54) Personal Care Product that Detects the Presence of Controlled Substances, and Methods of Use Thereof

(71) Applicant: Larik Biotech, Orlando, FL (US)

(72) Inventors: Krista Thompson, Orlando, FL (US); Lawrence Nobel, Orlando, FL (US)

(21) Appl. No.: 19/074,369

(22) Filed: Mar. 8, 2025

(51) Int. Cl. A61K 8/92; A61Q 19/00; G01N 33/94

(52) U.S. Cl. CPC A61K 8/92; A61Q 19/001; G01N 33/94; A61K 2800/45; A61K 2800/5922; A61K 2800/87

(57) ABSTRACT

Embodiments of the present invention comprise a personal care product that detects for controlled substances, and methods of use thereof. The personal care product may comprise a balm configured within a container. The balm may comprise ingredients common to other balms in the art such but not limited to waxes, surfactants, and emollients. The balm may further comprise cobalt thiocyanate for detecting cocaine, a pH indicator for detecting gamma hydroxybutyrate, a molecularly imprinted polymer for detecting flunitrazepam, a reactive polymer for detecting fentanyl, a chromogenic agent for detecting for flunitrazepam, ketamine, and/or scopolamine, and a Marquis reagent for detecting for 3,4 methylenedioxymethamphetamine and/or methamphetamine. Before or after ingesting a controlled substance, a user may apply the balm to their lips, which causes the balm to change color when reacting with the controlled substance on the user's lips. The user or a bystander may notice this color change.

### BACKGROUND OF THE INVENTION

The present invention relates to the detection of controlled substances. Controlled substances such as but not limited to flunitrazepam, gamma hydroxybutyrate, and ketamine are often used in sexual assaults. Sexual predators will put an amount of a controlled substance into a victim's food or beverage when the victim is not looking, and the victim will unknowingly ingest the controlled substance. The controlled substance generally has an incapacitating effect on the victim, making it easier for the sexual predator to take advantage of the victim.

Products exist that test for controlled substances. These products contain chemicals that undergo reactions when coming into contact with controlled substances. However, these products are often indiscreetly designed such that it is obvious that someone is using the product to test for a controlled substance. This indiscreetness may not be desirable in a social setting such as a bar or nightclub. Furthermore, these products often require users to bring the products in direct contact with a food or beverage, further decreasing the discreetness of the products.

A further drawback of common controlled substance testing products is that they often test for only a few different controlled substances. While flunitrazepam and gamma hydroxybutyrate are the two controlled substances most commonly associated with sexual assault, a multitude of other controlled substances may be used by sexual assault predators such as but not limited to cocaine, scopolamine, and 3,4-methylenedioxymethamphetamine. Testing for a multitude of controlled substances would require carrying multiple controlled substance testing products on one's person and testing a food or beverage with each controlled substance testing product.

### SUMMARY OF THE INVENTION

Embodiments of the present invention comprise a personal care product. The personal care product may comprise a container with a container open end and a container closed end. A balm may be configured within the container and accessible via the container open end. The balm may be configured to extend from and retract into the container via the container open end.

The balm may comprise a wax. The wax may be beeswax, candelilla wax, or a combination of both. The wax may further be any plant-based or animal-based wax. The wax may make up 15%-45%, inclusive, of the balm by weight.

The balm may comprise a surfactant. The surfactant may be coconut oil or any other substance commonly used as a surfactant in personal care products. The surfactant may make up 10%-30%, inclusive, of the balm by weight. The balm may comprise a viscosity controller. The viscosity controller may be kokum butter or any other substance commonly used as a viscosity controller in personal care products. The viscosity controller may make up 10%-30%, inclusive, of the balm by weight. The balm may comprise an emollient. The emollient may be isoamyl laurate or any other substance commonly used as an emollient in personal care products. The emollient may make up 15% or less of the balm by weight. The balm may comprise an anti-irritant. The anti-irritant may be jojoba oil or any other substance commonly used as an anti-irritant in personal care products. The anti-irritant may make up 10%-30%, inclusive, of the balm by weight. The balm may comprise a fragrance.

The balm may comprise cobalt thiocyanate that may change color when coming into contact with cocaine. The balm may comprise one or more pH indicators that may change color when coming into contact with gamma hydroxybutyrate. The balm may comprise one or more molecularly imprinted polymers that may change color when coming into contact with flunitrazepam. The balm may comprise one or more reactive polymers that may change color when coming into contact with fentanyl. The balm may comprise one or more chromogenic agents that may change color when coming into contact with flunitrazepam, ketamine, scopolamine, or any combination thereof. The balm may comprise a Marquis reagent that may change color when coming into contact with 3,4-methylenedioxymethamphetamine, methamphetamine, or a combination thereof. The cobalt thiocyanate, the one or more pH indicators, the one or more molecularly imprinted polymers, the one or more reactive polymers, the one or more chromogenic agents, and the Marquis reagent together may make up 5%-25%, inclusive, of the balm by weight.

The balm may be applied directly to a user's lips form the container before after the user ingests a controlled substance. The balm within the container and the balm applied to the user's lips may change color based on the controlled substance ingested by the user.

Embodiments of the present invention may also comprise methods of detecting for controlled substances. These methods may utilize the personal care product of the present invention. A balm may be provided. The balm may be the same balm described previously herein with the same ingredients and color-changing functionality. A container with an open and a closed end may be provided. The balm may be configured within the container such that the balm is accessible via the container open end. A user may apply the balm directly to the user's lips from the container before or after the user has ingested a controlled substance. The balm on the user's lips and the balm within the container may react with the controlled substance and may change color based on the specific controlled substance ingested by the user.

In some embodiments of the methods, a bystander may view the color change of the balm on the user's lips and may notify the user of this color change. In other embodiments of the methods, the user may view the color change of the balm within the container and may thereby be alerted that they have ingested a controlled substance.

### BRIEF DESCRIPTION OF DRAWINGS

FIG. 1 shows a perspective view of a personal care product according to embodiments of the present invention.

FIG. 2 shows a front view of the personal care product of FIG. 1 being applied to a user's lips.

FIG. 3 shows a flowchart of a method for detecting the presence of a controlled substance according to embodiments of the present invention.

FIG. 4 shows a flowchart of a method for detecting the presence of a controlled substance according to embodiments of the present invention.

### DETAILED DESCRIPTION OF THE INVENTION

The description provided herein describes example embodiments of the present invention and is not intended to limit the present invention to any particular embodiment, feature, component, material, chemical composition, use, functionality, method steps, or any other property. Furthermore, the drawings provided herein show example embodiments of the present invention and are not intended to limit the present invention to any particular embodiment, feature, component, material, chemical composition, use, functionality, method steps, or any other property.

Throughout this disclosure, the terms “substance” and “controlled substance” are both used. As used herein, the term “substance” refers to any chemical makeup, which could be harmful to humans, beneficial to humans, or neither. Per this definition, examples of “substances” include but are not limited to water, hydrogen peroxide, cocoa butter, cyanide, etc. As used herein, the term “controlled substance” refers to a substance that is generally considered harmful to humans in large doses, but that may be ingested by humans for recreational or medicinal purposes. A “controlled substance” may be a substance wherein its use, possession, and/or manufacturing is regulated. Per these definitions, examples of “controlled substances” include but are not limited to flunitrazepam, methamphetamine, hydrocodone, ketamine, ethanol, etc. Per these definitions, all “controlled substances” are “substances”, but not all “substances” are “controlled substances”.

Various ingredients of the balm of the present invention are described herein. These ingredients may be described in the singular form. This is not intended to limit the present invention to comprising only one type of those ingredients. For example, the balm of the present invention is described as having “a wax” herein. In some embodiments, a single type of wax such as beeswax may be used to form the balm. However, in other embodiments, multiple types of waxes such as both beeswax and candelilla wax may be used to form the balm. This logic may be applied to other ingredients of the balm described herein such as but not limited to the chromogenic agent, the molecularly imprinted polymer, the surfactant, the emollient, etc.

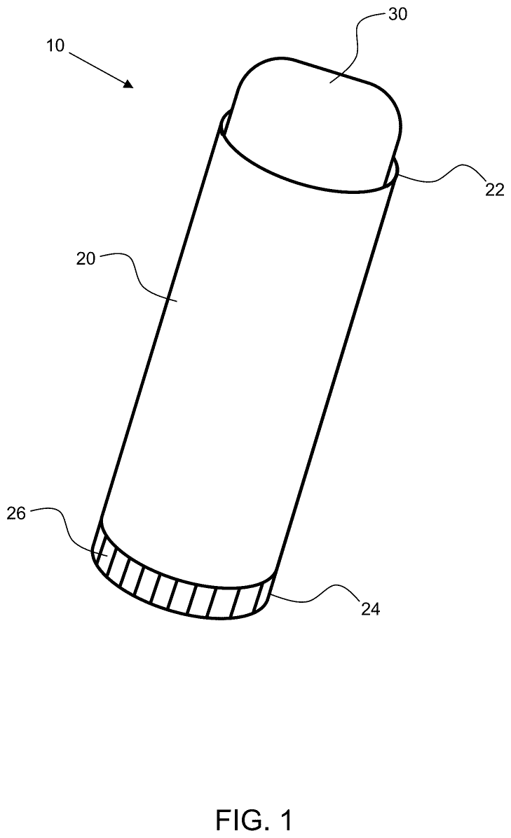

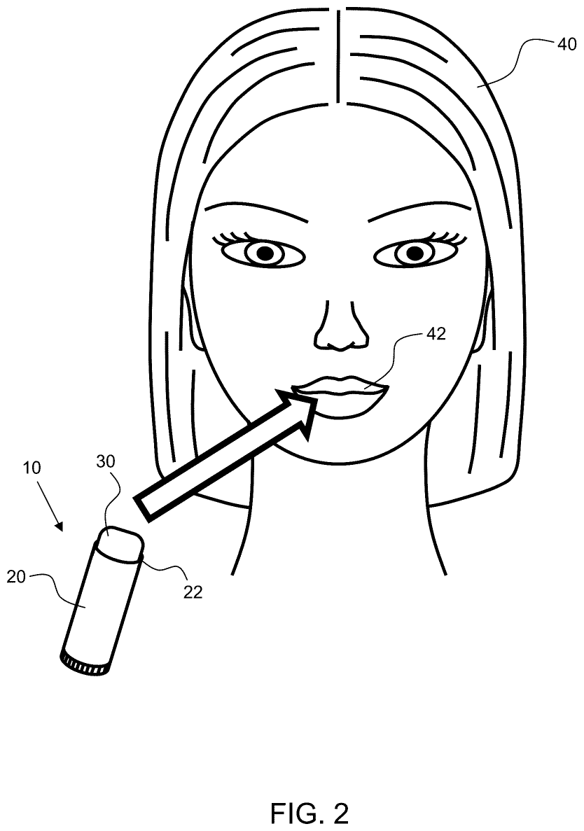

As shown in FIGS. 1 and 2, a personal care product 10 comprises a balm 30 contained within a container 20. The container 20 has a container open end 22 and a container closed end 24. An actuator controller 26 is configured at the container closed end 24. When manipulated, the actuator controller 26 causes the balm 30 to extend further out from the container 20 or to retract further into the container 20 depending on how the actuator controller 26 is manipulated. For example, the actuator controller 26 may be twisted clockwise to cause the balm 30 to extend further out from the container 20 and counterclockwise to cause the balm 30 to retract further into the container 20. In another example, the actuator controller 26 may be pressed towards the container open end 22 to cause the balm 30 to extend further out form the container 20 and may be pulled away from the container open end 22 to cause the balm 30 to retract further into the container 20.

The portion of the balm 30 that extends from the container open end 22 may be applied to the lips 42 of a user 40. The user 40 may apply the balm 30 to their lips 42 before or after ingesting a substance that may contain a controlled substance for which the personal care product 10 tests, thereby causing the controlled substance to make contact with the balm 30 in the container 20 and also the balm on the user's lips 42.

The balm may have a consistency similar to common lip balms that exist in the art. This means that the balm may have a viscosity at 70 degrees Celsius within the range of 0.005 Pa-s-15 Pa-s, inclusive. The balm may comprise a wax. The wax may make up 15%-45%, inclusive, of the balm by weight, and may provide the physical structure of the balm due to its extremely high viscosity. The wax may be a nonpolar lipid. The wax may be an ester of saturated fatty acids and/or alcohols. Examples of waxes that may be used to form the balm include but are not limited to beeswax and candelilla wax. In some embodiments, candelilla wax is preferred since it is produced by a plant rather than an animal, and therefore may appeal to users that avoid purchasing products that contain animal-derived substances.

The balm may comprise a surfactant. The surfactant may act as an emulsifier for mixing the oil-based ingredients and water-based ingredients of the balm. This may allow the balm to have a uniform consistency, and may further allow the balm to spread smoothly over a user's lips when the balm is applied to the user's lips. The surfactant may make up 10%-30%, inclusive, of the balm by weight. An example of a surfactant that may be used to form the balm includes but is not limited to coconut oil.

The balm may comprise a viscosity controller. The viscosity controller may increase or decrease the overall viscosity of the balm depending on the exact viscosity controller used and the amount of viscosity controller added. The viscosity controller may make up 10%-30%, inclusive, of the balm by weight. Examples of a viscosity controllers that may be used to form the balm include but are not limited to kokum butter and cocoa butter. Both of these substances have lower viscosities than beeswax or candelilla wax, and therefore adding more kokum butter or cocoa butter would lower the overall viscosity of the balm.

The balm may comprise an anti-irritant. The anti-irritant may cause the balm to deliver a pleasant sensation to the user's lips when the balm is applied to the user's lips. The anti-irritant may make up 10%-30%, inclusive, of the balm by weight. An example of an anti-irritant that may be used to form the balm may include but is not limited to jojoba oil.

The balm may comprise an emollient. The emollient may be a hydrophobic substance that prevents water loss from the user's lips when the balm is applied to the user's lips. The emollient may form a barrier on the user's lips that prevents water from evaporating from the user's lips, therefore keeping the user's lips moisturized. This barrier may also prevent tannins in products such as wine and coffee from staining the user's lips. This may allow for a more aesthetic look of the user's lips, and may also enhance the effectiveness of the personal care product at causing the user's lips to portray the desired color change when a user ingests a controlled substance. The emollient may make up 15% or less of the balm by weight. An example of an emollient used to form the balm may include but is not limited to isoamyl laurate. In some embodiments, substances that serve as other ingredients such as kokum butter, cocoa butter, and jojoba oil may also be used as emollients due to their hydrophobic nature.

The balm may comprise a fragrance. The fragrance may emit a pleasant smell from the balm. The fragrance may be oil-based or water-based. The fragrance may make up 5% or less of the balm by weight. Examples of fragrances used to form the balm include but are not limited to essential oils, which are concentrated extracts of plants that are generally known to have aromas considered pleasant by humans.

The balm may comprise a moisturizer. The Moisturizer may be a hydrophilic substance that causes the user's lips to absorb water, or a hydrophobic substance that prevents water from evaporating from the user's lips. The moisturizer may make up 15% or less of the balm by weight. An example of a moisturizer used to form the balm may include but is not limited to meadowfoam seed oil.

The moisturizer may also be used as a carrier for other oil-based ingredients of the balm such as the fragrance. In some embodiments, other ingredients such as the viscosity controller may also serve as a moisturizer. For example, kokum butter and cocoa butter are known in the art of cosmetics to have moisturizing properties when applied to one's skin. Therefore, either of these substances may be used as both a viscosity controller and a moisturizer. Both kokum butter and cocoa butter are oil-based and may therefore also be used as carriers for other oil-based ingredients of the balm such as the fragrance. In another example, isoamyl laurate may be used as an emollient, thereby aiding in moisturization of the user's lips, and thereby acting as a moisturizer. Isoamyl laurate is oil-based and may therefore also be used as a carrier for other oil-based ingredients of the balm such as the fragrance.

In addition to ingredients that form the consistency, texture, and aroma of the balm, the balm may comprise other ingredients that detect the presence of a controlled substance. The balm may comprise cobalt thiocyanate. Cobalt thiocyanate is known for its use in detecting the presence of cocaine. Cobalt thiocyanate is colorless, but turns bright blue when brought into contact with cocaine. Therefore, if the user of the personal care device unknowingly ingests cocaine and has cocaine residue on their lips, the balm will turn bright blue when the user applies the balm to their lips.

The balm may comprise a pH indicator. pH indicators are substances that change color when coming into contact with another substance that has a specific pH range. pH indicators are often used to differentiate between acids and bases. The pH indicator of the present invention may change color when coming into contact with gamma hydroxybutyrate (GHB). In some embodiments, the pH indicator may be yellow and may turn green-blue when coming into contact with GHB. In some embodiments, the specific pH indicator used to detect for GHB is bromocresol purple. A pH indicator is chosen to detect for GHB since GHB has a pH that often falls within the range of 4-7, inclusive.

The balm may comprise a molecularly imprinted polymer (MIP). MIPs are synthetic polymers that are formed using molecules of a target substance. After forming the MIP, the molecules of the target substance are removed, leaving cavities in the MIP that are designed to bind with molecules of the target substance. Therefore, when an MIP comes into contact with its target substance, the molecules of the MIP bind to the molecules of the target substance and cause a reaction. This reaction may be a change in color. The MIP of the present invention is formed using flunitrazepam molecules. In some embodiments, the MIP of the present invention may be pale yellow and may turn purple when coming into contact with flunitrazepam. A chromogenic agent may be used in parallel with the MIP to cause this color change to occur.

The balm may comprise a reactive polymer. The reactive polymer may undergo a chemical reaction when coming into contact with a target substance. The reactive polymer may be natural or synthetic. In some embodiments, the reactive polymer may be an MIP, though this is not necessarily the case in all embodiments. The reactive polymer of the present invention may undergo a chemical reaction when coming into contact with fentanyl. The reactive polymer of the present invention may be colorless and may turn a deep red when undergoing the chemical reaction that occurs when coming into contact with fentanyl. In some embodiments, this deep red color change may be aided by a dye that may be activated by the chemical reaction caused by the reactive polymer coming into contact with fentanyl.

The balm may comprise a chromogenic agent. A chromogenic agent is a chemical that undergoes a color change when reacting with a target substance. A single chromogenic agent may be used in the balm of the present invention to detect for multiple different controlled substances. Alternatively, multiple chromogenic agents may be used in the balm of the present invention, wherein each of the multiple chromogenic agents is used to detect for a specific controlled substance. A chromogenic agent of the present invention may undergo a color change from colorless to pink-purple when coming into contact with ketamine. A chromogenic agent of the present invention may undergo a color change from colorless to light brown-red when coming into contact with scopolamine. A chromogenic agent of the present invention may undergo a color change from colorless or yellow to purple when coming into contact with flunitrazepam. The chromogenic agent(s) of the present invention may be used in tandem with other chemicals such as the pH indicator, MIP, and reactive polymer to detect for the controlled substances targeted by those chemicals. When used in tandem with the pH indicator, MIP, and/or reactive polymer, the chromogenic agent(s) may allow for the desired color changes to occur that allow the pH indicator to detect GHB, that allow for the MIP to detect flunitrazepam, and that allow for the reactive polymer to detect fentanyl.

The balm may comprise a Marquis reagent (also commonly known as “the Marquis reagent”). The Marquis reagent is made with formaldehyde and sulfuric acid, and is commonly used to test for various controlled substances such as but not limited to 3,4-methylenedioxymethamphetamine (MDMA) and methamphetamine. When used in the present invention, the Marquis reagent may cause the balm to turn green when coming into contact with MDMA, and orange-brown when coming into contact with methamphetamine.

The balm is described herein as changing colors when brought into contact with the user's lips if the user has residue of a controlled substance for which the balm detects on their lips. When this occurs, both the balm within the container of the personal care device and the balm that was applied to the user's lips may change color. Therefore, a bystander may become aware that the user has ingested a controlled substance by viewing the color change of the user's lips after applying the balm. The user may also view the balm within the container for a color change after applying the balm to their lips, and may become aware that they have ingested a controlled substance based on the color change of the balm within the container. The chemicals chosen for detection of the various controlled substances described herein function well together in the balm since they cause the balm to change to different colors depending on the controlled substance that is detected. For example, the balm may turn green-blue when coming into contact with GHB, purple when coming into contact with flunitrazepam, pink-purple when coming into contact with ketamine, bright blue when coming into contact with cocaine, light brown-red when coming into contact with scopolamine, deep red when coming into contact with fentanyl, green when coming into contact with MDMA, and orange-brown when coming into contact with methamphetamine. The balm's natural color may be colorless or a shade of yellow.

While other chemicals may be used to detect for the specific controlled substances described herein, the specific chemicals utilized by the present invention allow for easy differentiation between controlled substances based on the different color changes that may occur. This allows a user or a bystander to better determine which controlled substance that the user ingested so they may react accordingly. For example, naloxone is commonly used as a treatment for fentanyl overdoses, but is ineffective against non-opioid drugs such as flunitrazepam. Therefore, it is beneficial that the balm is configured to change one color when coming into contact with fentanyl and another color when coming into contact with flunitrazepam so that the correct care may be administered to the user depending on which controlled substance the user ingests.

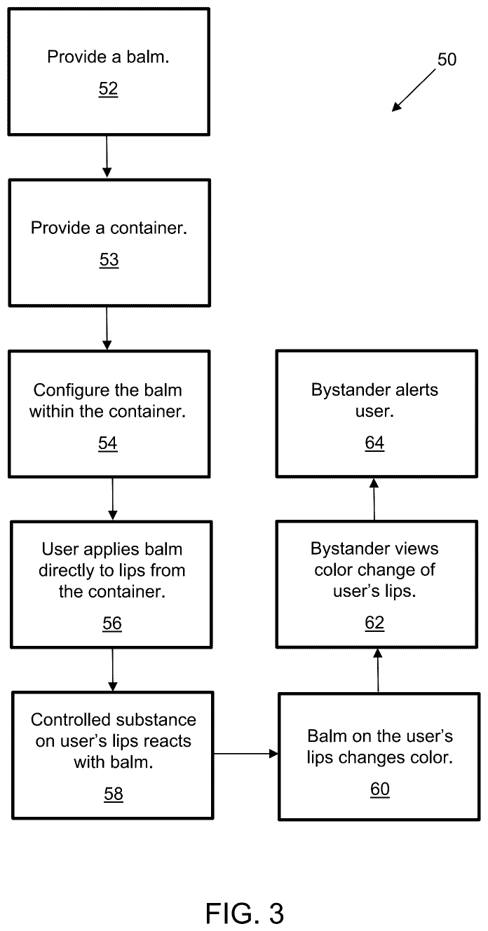

As shown in FIG. 3, the personal care product of FIGS. 1 and 2 may be used in a method for detecting a controlled substance 50. In the method 50 shown in FIG. 3, a balm is provided 52 and a container 53 is provided. The balm is configured within the container 54. The balm may be configured in the container such that a portion of the balm extends out of the container through the container open end. A user applies the balm directly to the user's lips from the container 56. If the user has ingested a controlled substance and amounts of the controlled substance are present on the user's lips, then the controlled substance on the user's lips reacts with the balm 58. This causes the balm on the user's lips to change color 60. A bystander notices the color change of the user's lips 62, and then the bystander alerts the user of this color change 64. The bystander may be any person that is not the user or the person that provided the controlled substance to the user. The bystander may be a companion of the user or a worker at a bar/restaurant/nightclub.

In some embodiments of the method 50, bystanders may be trained on the use of the personal care device. During this training, bystanders may learn which color change is associated with certain controlled substances. Bystanders may also learn appropriate care measures to be taken depending on which controlled substance is ingested by the user. For example, bystanders may be trained that if a user's lips suddenly turn pink-purple, this is sign that the user has ingested ketamine. Bystanders may be trained that there are no common antidotes for ketamine and that they should call emergency services in this situation.

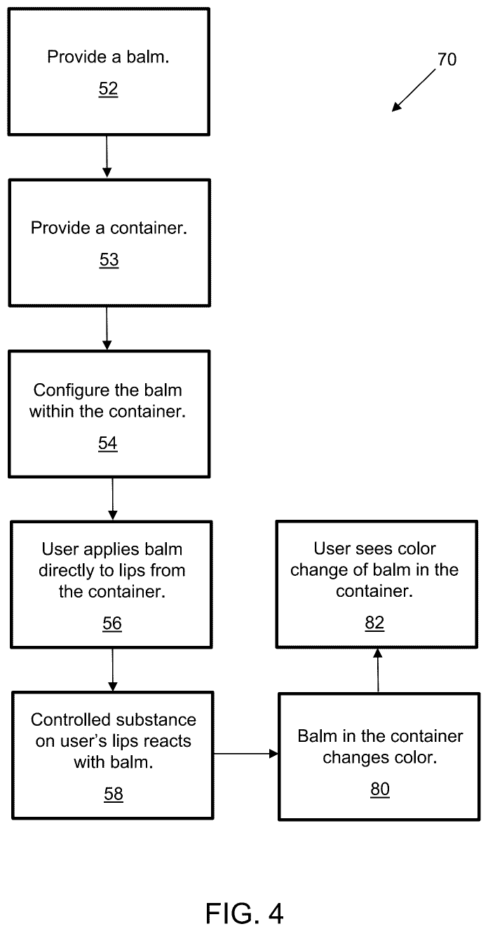

As shown in FIG. 4, the personal care product of FIGS. 1 and 2 may be used in another method for detecting a controlled substance 70. Similar to the method 50 shown in FIG. 3, a balm is provided 52 and a container 53 is provided in the method 70 shown in FIG. 4. The balm is configured within the container 54, and a user applies the balm directly to the user's lips from the container 56. If the user has ingested a controlled substance and amounts of the controlled substance are present on the user's lips, then the controlled substance on the user's lips reacts with the balm 58. In the method 70 shown in FIG. 4, the balm in the container changes color 80 after reacting with the controlled substance. This allows the user themself to view the color change in the balm within the container 82. This alerts the user that they have ingested a controlled substance and that they should seek help.

The balm may be applied to the user's lips before or after the user ingests a controlled substance. In embodiments wherein the balm is applied before the user ingests a controlled substance, the user may consume a food or beverage containing the controlled substance. The controlled substance in that food or beverage may touch the user's lips to which the balm has already been applied, and the controlled substance may react with the balm on the user's lips to change the color of the balm. In embodiments wherein the balm is applied before the user ingests the controlled substance, only the balm on the user's lips and not the balm in the container may change color, since the balm in the container does not come into direct contact with the controlled substance.

In embodiments wherein the balm is applied after the user ingests a controlled substance, amounts of the controlled substance may be present on the user's lips. When the user applies the balm to their lips, the balm may react with the amounts of the controlled substance on the user's lips to change the color of the balm. In embodiments wherein the balm is applied after the user ingests a controlled substance, both the balm on the user's lips and the balm in the container may change color since both the balm on the user's lips and the balm in the container come into direct contact with the controlled substance.

1. A personal care product comprising:
a container comprising a container open end and a container closed end;
a balm configured within the container and accessible via the container open end, the balm being applied directly to a user's lips from the container after the user ingests a controlled substance, the balm comprising:
a wax;
cobalt thiocyanate that changes color when coming into contact with cocaine;
a pH indicator that changes color when coming into contact with gamma hydroxybutyrate;
a molecularly imprinted polymer that changes color when coming into contact with flunitrazepam;
a reactive polymer that changes color when coming into contact with fentanyl;
a chromogenic agent that changes color when coming into contact with at least one of flunitrazepam, ketamine, or scopolamine; and
a Marquis reagent that changes color when coming into contact with at least one of 3,4-methylenedioxymethamphetamine or methamphetamine,
wherein the application of the balm to the user's lips before the user ingests the controlled substance causes the balm on the user's lips to change color based on the controlled substance ingested by the user,
and wherein the application of the balm to the user's lips after the user ingests the controlled substance causes the balm within the container and the balm applied the user's lips to change color based on the controlled substance ingested by the user.

2. The personal care product of claim 1, wherein the balm further comprises a fragrance.

3. The personal care product of claim 1, wherein the balm further comprises a surfactant.

4. The personal care product of claim 3, wherein the surfactant is coconut oil.

5. The personal care product of claim 1, wherein the balm further comprises a viscosity controller.

6. The personal care product of claim 5, wherein the viscosity controller is kokum butter.

7. The personal care product of claim 1, wherein the balm further comprises an emollient.

8. The personal care product of claim 7, wherein the emollient is isoamyl laurate.

9. The personal care product of claim 1, wherein the balm further comprises an anti-irritant.

10. The personal care product of claim 9, wherein the anti-irritant is jojoba oil.

11. The personal care product of claim 1, wherein the wax is beeswax.

12. The personal care product of claim 1, wherein the wax is candelilla wax.

13. The personal care product of claim 1, wherein the wax makes up 15%-45%, inclusive, of the balm by weight.

14. The personal care product of claim 1, wherein the cobalt thiocyanate, the pH indicator, the molecularly imprinted polymer, the reactive polymer, the chromogenic agent, and the Marquis reagent together make up 5%-25%, inclusive, of the balm by weight.

15. The personal care product of claim 3, wherein the surfactant makes up 10%-30%, inclusive, of the balm by weight.

16. The personal care product of claim 5, wherein the viscosity controller makes up 10%-30%, inclusive, of the balm by weight.

17. The personal care product of claim 7, wherein the emollient makes up 15% or less of the balm by weight.

18. The personal care product of claim 9, wherein the anti-irritant makes up 10%-30%, inclusive, of the balm by weight.

19. A method of detecting the presence of a controlled substance, comprising:
providing a balm comprising:
a wax;
cobalt thiocyanate that changes color when coming into contact with cocaine;
a pH indicator that changes color when coming into contact with gamma hydroxybutyrate;
a molecularly imprinted polymer that changes color when coming into contact with flunitrazepam;
a reactive polymer that changes color when coming into contact with fentanyl;
a chromogenic agent that changes color when coming into contact with at least one of flunitrazepam, ketamine, or scopolamine;
a Marquis reagent that changes color when coming into contact with at least one of 3,4-methylenedioxymethamphetamine or methamphetamine;
providing a container comprising a container open end;
configuring the balm within the container whereby the balm is accessible via the container open end;
a user applying the balm directly to the user's lips from the container before the user ingests a controlled substance;
the user ingesting a controlled substance;
a bystander viewing the user's lips for a color change to indicate the presence of a controlled substance; and
the bystander notifying the user of the color change in the user's lips.

20. A method of detecting the presence of a controlled substance, comprising:
providing a balm comprising:
a wax;
cobalt thiocyanate that changes color when coming into contact with cocaine;
a pH indicator that changes color when coming into contact with gamma hydroxybutyrate;
a molecularly imprinted polymer that changes color when coming into contact with flunitrazepam;
a reactive polymer that changes color when coming into contact with fentanyl;
a chromogenic agent that changes color when coming into contact with at least one of flunitrazepam, ketamine, or scopolamine;
a Marquis reagent that changes color when coming into contact with at least one of 3,4-methylenedioxymethamphetamine or methamphetamine;
providing a container comprising a container open end;
configuring the balm within the container whereby the balm is accessible via the container open end;
a user applying the balm directly to the user's lips from the container after the user ingests a controlled substance; and
the user viewing the balm within the container for a color change to indicate the presence of a controlled substance.
